(19) United States
(12) Patent Application Publication
(10) Pub. No.: US 2021/0361750 A1
(43) Pub. Date: Nov. 25, 2021

(54) Anti-Viral Lactoferrin Facemask

(71) Applicant: Virustatic Ltd., Anglesey (GB)

(72) Inventor: Paul Francis Hope, Stockport (GB)

(21) Appl. No.: 17/322,815

(22) Filed: May 17, 2021

Related U.S. Application Data
(60) Provisional application No. 63/028,073, filed on May 21, 2020.

(51) Int. Cl. A61K 38/40; A01N 63/50; A01N 25/34; A62B 7/10; A62B 23/02; A41D 13/11

(52) U.S. Cl. CPC A61K 38/40; A01N 63/50; A41D 13/1192; A62B 7/10; A62B 23/02; A01N 25/34

(57) ABSTRACT

A breathable biological face covering comprising a fabric material and an anti-pathogen substance on the fabric material. The anti-pathogen substance comprises a lactoferrin protein. The amount of lactoferrin protein on the fabric material may be in the range of 0.6-1.5 grams/m² of fabric material. Another embodiment is a lactoferrin composition comprising an aqueous fluid at pH<7.0 and lactoferrin protein mixed in the aqueous fluid at a concentration of 0.6-1.5 grams per liter. This lactoferrin composition may be administered into the respiratory tract. This composition could be useful against respiratory microbial pathogen, such as influenza virus or coronavirus.

### TECHNICAL FIELD

This invention relates to treatments for respiratory viral infections.

### BACKGROUND

As the recent pandemic has demonstrated, respiratory viruses such as influenza and coronaviruses such as SARS-CoV-2 (severe acute respiratory syndrome coronavirus 2) can cause a significant global health burden. SARS-CoV-2 is the causative agent of COVID-19. Such respiratory viruses are generally transmitted by airborne aerosols and droplets from infected people emitted by breathing, speaking, and coughing. Even before the SARS-CoV-2 pandemic, various strains of influenza viruses have circulated at the pandemic level. The most recent pandemic-level influenza strain emerged in 2009, named influenza A H1N1pdm09 virus. It is estimated that between 2009-2018, this virus has caused at least 100 million cases with 900,000 hospitalizations and 75,000 deaths, even without pandemic-level spread, seasonal epidemics of influenza cause about 290,000-650,000 deaths each year. Thus, in addition to new coronaviruses, there is concern that new influenza viruses may evolve that cause higher mortality and morbidity.

The occurrence of these pandemics has also led to significant global economic, social, and political disruptions that have highlighted the need for strategies to reduce their spread. The most widely used strategy to combat the SARS-CoV-2 pandemic has been facemask use by the general public. However, facemasks are not completely effective in filtering out respiratory viruses. The filtration efficiency for facemasks can be as low as 5% for very small particles of <0.1 μm size. Thus, there is a need for improved facemasks that are more effective in trapping, capturing, neutralizing, capture, or otherwise protecting the user from microbial pathogens.

### SUMMARY

As used herein, “antiviral efficacy” means the ability to kill, neutralize, inactivate, capture, or otherwise reduce the amount or strength of the virus. As used herein, “anti-pathogen efficacy” means the ability to kill, neutralize, inactivate, capture, or otherwise reduce the amount or strength of the microbial pathogens (e.g. virus, bacteria, fungus). This antimicrobial efficacy could be used for the prevention or treatment of various microbial pathogen infections.

Biological Face Covering: In one aspect, this invention is a breathable biological face covering that comprises lactoferrin. This invention covers a variety of different types of face coverings. Examples of face coverings include facemasks, snoods, surgical masks, respirator masks, etc. Snoods that cover the lower face and neck may be particularly useful because conventional facemasks have leakage around the sides that compromise the filtration effectiveness.

The face covering comprises a fabric material and an anti-pathogen substance on the fabric material, wherein the anti-pathogen substance comprises a lactoferrin protein. Any suitable fabric material may be used, including those made of cotton, wool, silk, bamboo fibers, viscose, lyocell, cellulosic fibers, eucalyptus fibers, nylon, polyester, poly-lactic acid, acrylic, or rayon. The anti-pathogen substance works to kill, neutralize, inactivate, capture, or otherwise protect the user from pathogens. The anti-pathogen substance may further include other anti-pathogenic ingredients, such as silver, activated carbon, quaternary ammonium compounds, glycosaminoglycans, natural antiviral or antibacterial compounds, etc.

In some embodiments, the face covering comprises a fabric dye that is applied to the fabric material. See below section for more details. The face covering of this invention could be single-use and disposable, or it could be designed for multiple, repeated use; and in some cases, reusable and maintaining anti-pathogen efficacy even after laundry washings. This may be useful in reducing environmental waste.

Making of the Biological Face Covering: In another aspect, this invention is a method of making a breathable biological face covering. The method comprises applying lactoferrin to the fabric material. The lactoferrin is provided in an aqueous solution containing lactoferrin. In some embodiments, the lactoferrin concentration is 0.6-1.5 grams per liter; and in some cases, 0.75-1.25 grams per liter. The aqueous solution may be adjusted to a pH level in the range of <7.0; and in some cases, pH of 4.5-6.5.

The lactoferrin aqueous solution is applied to the fabric material. This could be performed by any suitable technique, such as spraying, soaking, dabbing, padding, rinse cycle of a washing machine, etc. In some embodiments, the resulting amount of lactoferrin applied to the fabric material is in the range of 0.6-1.5 grams/m² of fabric material; and in some cases, 0.75-1.25 grams/m².

The fabric material may be treated with a fabric dye. As such, in some embodiments, the method further comprises applying a fabric dye to the fabric material. In some embodiments, the fabric dye comprises a reactive dye compound which comprises a chromophore attached to substituent group(s) that are capable of directly reacting with the fiber substrate of the fabric material and form covalent bonds therewith. In some embodiments, the reactive dye compound comprises one or more sulfonic acid groups. In some embodiments, the reactive dye compound is anionic. Being anionic or having sulfonic acid group(s) could have the function of bonding to the cationic lactoferrin protein by electrostatic or ion exchange reaction bonding. The sulfonic acid groups would ionize {RSO₃H→RSO₃⁻+H⁺} during the dying process, become anionic, and bind to the cationic lactoferrin protein by electrostatic or ion exchange interaction.

The reactive dye compound would bond to the fabric material in a conventional fashion (e.g. by electrostatic or covalent bonding). Moreover, by being anionic or having sulfonic acid group(s), the reactive dye compound could also bond to the lactoferrin protein. Thus, the reactive dye compound could serve the special function of adhering the lactoferrin to the fabric material substrate, i.e. work as an intermediate coupling between the lactoferrin protein and the fabric material substrate.

In some embodiments, the reactive dye compound comprises an azo dye compound having the diazo functional group (—N═N—). In some cases, the azo dye compound is anionic. In some cases, the azo dye compound has one or more sulfonic acid groups. In some cases, the azo dye compound has multiple (two or more) sulfonic acid groups; for example, 2-5 sulfonic acid groups.

The method further comprises drying the fabric material. The drying temperature could be selected to avoid denaturing or damaging the lactoferrin protein. In some embodiments, the fabric material is dried at a temperature≤80° C. (e.g. a temperature in the range of 50-80° C.).

Lactoferrin Composition: In another aspect, this invention is a lactoferrin composition comprising an aqueous fluid at pH<7.0; and in some cases, pH 4.5-6.5. The composition further comprises lactoferrin protein. In some embodiments, the concentration is 0.6-1.5 grams per liter; and in some cases, 0.75-1.25 grams per liter. The composition is liquid and may have the form of any of the various types of liquid mixtures, such as a solution, suspension, emulsion, gel, sol, liquid foam, etc.

In some embodiments, the lactoferrin composition contains no other ingredients having a molecular weight of greater than 500 grams/mol, or essentially none thereof (less than 0.05 grams/L). This may be useful to ensure that the lactoferrin composition is free of ingredients that could interfere with the anti-pathogen effect of lactoferrin. However, the lactoferrin composition could contain other low-molecular weight substances that do not interfere with the anti-pathogen effect of lactoferrin.

In another aspect, this invention is a lactoferrin composition that is a powder material. This powder material could be made into a dissolvable tablet or an effervescent tablet for use in a spray or in a washing machine cycle. The lactoferrin powder could be at any suitable pH level. In some embodiments, the lactoferrin powder is at pH<7.0; and in some cases, pH 4.5-6.5. The powder could be made by spray drying, freeze drying, or possibly ultrasound drying to form particle sizes between 0.1-10 μm. The dose concentration could vary between 100-1,000 μg. In some embodiments, the powder lactoferrin composition contains no other ingredients having a molecular weight of greater than 500 grams/mol, or essentially none thereof (less than 3 wt %). This may be useful to ensure that the lactoferrin composition is free of ingredients that could interfere with the anti-pathogen effect of lactoferrin. However, the lactoferrin composition could contain other low-molecular weight substances that do not interfere with the anti-pathogen effect of lactoferrin.

Respiratory Route of Delivery: The lactoferrin composition (in either in an aqueous or powder form) may be delivered via the respiratory route such that the lactoferrin composition is deposited on respiratory tract lining. Any suitable type of respiratory device may be used to provide respiratory delivery of the lactoferrin composition. As such, in some embodiments, this invention is a respiratory device containing the lactoferrin composition. Examples of respiratory devices that could be used include inhalers (e.g. pressurized metered-dose inhaler), respirators, aerosolizers, nasal sprays, nebulizers, pipettes, squeeze bottles, or squirt tubes. In some embodiments, the respiratory device is a multi-use product and contains 3-20 mL volume of the lactoferrin composition. In some embodiments, the respiratory device is a single-use product and contains less than 1.5 mL volume of the lactoferrin composition. In some embodiments, the respiratory device does not contain any propellant (see explanation below) for forced spraying of the lactoferrin composition.

Treatment/Prevention: In another aspect, this invention is a method of protecting against microbial pathogens or treatment thereof. The microbial pathogens could be viruses, bacteria, fungi, etc. Examples of viruses include influenza and coronaviruses, such as the one recently identified as SARS-CoV-2 causing COVID-19 disease. Examples of bacterial pathogens include Streptococcus pneumoniae, Haemophilus influenzae, Pseudomonas aeruginosa, Staphylococcus aureus, group A streptococcus, and bacteria responsible for hospital-acquired bacterial infections.

In one embodiment, the method comprises wearing a biological face covering of the invention. The lactoferrin protein captures pathogens that are breathed-out or breathed-in by the user. Thus, the pathogens are trapped onto the fabric material. This trapping of pathogens may serve a dual purpose of protecting the user and also protecting others from an infected user.

The anti-pathogen effect of the biological face covering may be durable after laundry washing. In some embodiments, the method further comprises washing the biological face covering with water and optionally detergent. After this laundering, the biological face covering retains at least 70% of the anti-pathogen effect compared to a fresh, never-washed biological face covering. The wash temperature may be at least 21° C. is, but not more than 50° C. (i.e. 21-50° C.). In some embodiments, the temperature is at ambient room temperature. In some embodiments, the biological face covering retains at least 70% of the anti-pathogen effect after three washes; and in some cases, after five washes.

In another embodiment, the method comprises administering the lactoferrin composition of this invention into the user's respiratory tract. The lactoferrin composition may be deposited in one or more of the user's nostrils, nasopharynx, sinuses, trachea, bronchi, lungs, upper airways, lower airways, or central airways. The lactoferrin composition may be self-administered by the user or by someone else (e.g. caretaker, spouse, physician, nurse, therapist, etc.). In some embodiments, the lactoferrin composition is deposited only in the upper airways (e.g. nasopharynx, sinuses) and avoid depositing in the central airways (trachea and main-stem bronchi) or lungs. This may be done by not using any spray propellants with the lactoferrin composition (see explanation above).

Other Uses: The lactoferrin composition may also be incorporated into other types of devices, appliances, wearable articles, linens, etc. For example, the lactoferrin composition could be used in air conditioning units, blood filtration components, surface coverings for beds and other furniture, body coverings, gloves, wound dressings, socks, mastitis cups, tampons, and covers for windows or doors of buildings. This patent application also incorporates by reference the disclosure of U.S. Pat. No. 10,238,109 (issued 26 Mar. 2019; Paul Hope).

### BRIEF DESCRIPTION OF THE DRAWINGS

FIGS. 1A-1F show the viral inhibitory effect of various concentrations of lactoferrin across different pH levels. FIG. 1A shows the inhibitory effect at the highest lactoferrin concentration of 10 mg/mL. FIG. 1B is 5 mg/mL; FIG. 1C is 2.5 mg/mL; FIG. 1D is 1.25 mg/mL; FIG. 1E is 0.613 mg/mL; and FIG. 1F is 0.313 mg/mL.

FIGS. 2A-2D show the viral inhibitory effect at various pH levels across different concentrations of lactoferrin exposure. FIG. 2A shows the results at pH 4; FIG. 2B shows the results at pH 5; FIG. 2C shows the results at pH 6; FIG. 2D shows the results at pH 7.

FIG. 3 shows the cumulative percentage reduction of H1N1 titers from Viruferrin-treated fabric samples collected at 10 time points, ranging from 0 to 1440 minutes (24 hours).

FIG. 4 shows the antiviral activity against H1N1 by Viruferrin-treated fabric samples collected at various time points.

FIG. 5 shows the cumulative percentage reduction of SARS-CoV-2 titers from Viruferrin-treated fabric samples collected at 10 time points, ranging from 0 to 1440 minutes (24 hours).

FIG. 6 shows the antiviral activity against SARS-CoV-2 by Viruferrin-treated fabric samples collected at various time points.

FIG. 7 shows the viral titer of each well (virus remaining after the fabric wipe) using the microneutralization CPE-based assay.

FIG. 8 shows the capture rate of virus expressed as a percentage based upon the viral titer of the starting inoculum.

### DETAILED DESCRIPTION OF EXAMPLE EMBODIMENTS

SARS-CoV-2 enters the target cell mainly by attaching to the angiotensin converting enzyme 2 (ACE2) on the cell surface. The spike protein of SARS-CoV-2 mediates entry into human cells through pH-dependent endocytosis by interacting with ACE2 through its receptor-binding domain (RBD) in a dose dependent fashion. See Yang et al, “pH-dependent entry of severe acute respiratory syndrome coronavirus is mediated by the spike glycoprotein and enhanced by dendritic cell transfer through DC-SIGN” (2004) J Virol. <doi:10.1128/JVI.78.11.5642-5650.2004>. SARS-CoV-2 entry also involves initial priming of the spike protein by transmembrane protease, serine 2 (TMPRSS2). See Ou et al, “Characterization of spike glycoprotein of SARS-CoV-2 on virus entry and its immune cross-reactivity with SARS-CoV” (2020) Nat Commun. <doi: 10.1038/s41467-020-15562-9>; and Djomkam et al, “Commentary: SARS-CoV-2 Cell Entry Depends on ACE2 and TMPRSS2 and Is Blocked by a Clinically Proven Protease Inhibitor” (2020) Front Oncol. <doi: 10.3389/fonc.2020.01448>.

The SARS-CoV-2 spike protein is also recognized in a glycan-dependent manner by multiple innate immune cell receptors, making innate immune cells susceptible to SARS-CoV-2 infection. See Gao et al, “SARS-CoV-2 Spike Protein Interacts with Multiple Innate Immune Receptors” (2020) bioRxiv <doi: 10.1101/2020 0.07.29.227462>. In addition to this, the scientific literature points to various other factors that may be involved in SARS-CoV-2 infection process, such as lysosomal peptidase cathepsin L, hepsin, human airway trypsin-like protease (HAT), CD209L/L-SIGN, and CD209/DC-SIGN.

Lactoferrin is known to have both direct and indirect interference with the infection process for many viruses. As an example, lactoferrin has been shown to inhibit respiratory viruses such as influenza and other viruses. See Superti et al, “Bovine Lactoferrin Prevents Influenza A Virus Infection by Interfering with the Fusogenic Function of Viral Hemagglutinin” (2019) Viruses <doi: 10.3390/v11010051>. Lactoferrin blocks influenza infection by hindering viral adsorption and internalization into cells through specific binding to both virus-cell receptors and viral particles. See Ammendolia et al, “Bovine lactoferrin-derived peptides as novel broad-spectrum inhibitors of influenza virus” (2012) Pathogens & Global Health <doi: 10.1179/2047773212Y.0000000004>.

With regards to coronaviruses, lactoferrin has a direct effect on the binding of the virus to the cell. See Lang et al, “Inhibition of SARS Pseudovirus Cell Entry by Lactoferrin Binding to Heparan Sulfate Proteoglycans” (2011) PloS One <doi: 10.1371/journal.pone.0023710>. Lactoferrin has been shown to prevent human coronaviruses infecting the cell by binding to a heparan sulphate proteoglycan that is on the cell surface, thereby blocking an entry point for the virus. Lactoferrin may interfere with one or more of these virus interactions described above.

Lactoferrin has been shown to prevent SARS-CoV-2 infection, and also reduce the duration and severity of COVID-19 in affected patients. As such, lactoferrin has been proposed as a potential treatment for COVID-19. See Chang et al, “Lactoferrin as potential preventative and adjunct treatment for COVID-19” (2020) Int J Antimicrobial Agents <doi: 10.1016/j. ijantimicag.2020.106118>. While the exact mechanism for inhibition of SARS-CoV-2 by lactoferrin is unknown, it has been shown to inhibit viral entry for related-virus SARS-CoV either through virus-binding or cell surface molecule binding. See Mann et al, “The potential of lactoferrin, ovotransferrin and lysozyme as antiviral and immune-modulating agents in COVID-19” (2020) Future Virol. <doi: 10.2217/fv1-2020-0170>; Campione et al, “Lactoferrin as potential supplementary nutraceutical agent in COVID-19 patients: In vitro and in vivo preliminary evidences” (2020) bioRxiv <doi: 10.1101/2020.08.11.244996>; and Peroni et al, “Viral infections: Lactoferrin, a further arrow in the quiver of prevention” (2020) J. Pediatr. Neonatal Individ. Med. <doi: 10.7363/090142>. In clinical trials where lactoferrin is used as an antiviral, it has been shown to reduce the severity and duration of disease in patients with SARS-CoV-2 within 4-5 days after oral administration and prevent infection in people exposed to SARS-CoV-2. See Serrano et al, “Liposomal lactoferrin as potential preventative and cure for COVID-19” (2020) Int J Res Health Sci. <doi: 10.5530/ijrhs.8.1.3>.

Beneficial Effects of Lactoferrin: Lactoferrin has been shown to have antibacterial, antifungal, and antiviral effects (both indirectly and directly), and assists in immune defense by binding to a wide variety of pathogens. See Kell et al, “The Biology of Lactoferrin, an Iron-Binding Protein That Can Help Defend Against Viruses and Bacteria” (2020) Front Immun. <doi:10.3389/fimmu.2020.01221>. Reduced lactoferrin levels correlates with severity and morbidity of COVID-19, and also other virus infections such as influenza. As such, administration of lactoferrin may be especially useful in people who have reduced levels, such as certain ethnic populations, elderly, and diabetics.

Antiviral Effects of Lactoferrin: Lactoferrin has antiviral efficacy against a wide variety of viruses. See Waarts et al, “Antiviral activity of human lactoferrin: inhibition of alphavirus interaction with heparan sulfate” (2005) Virology <doi: 10.1016/j.viro1.2005.01.010>. There are a variety of proposed mechanisms for lactoferrin's antiviral effects. For example, one possible mechanism is that lactoferrin targets against initial viral entry. This invention encompasses any possible mechanism for lactoferrin's antiviral efficacy.

Antibacterial Effects of Lactoferrin: Secondary bacterial infections often occur with viral pneumonia (i.e. coinfection/superinfection). See Manohar et al, “Secondary Bacterial Infections in Patients With Viral Pneumonia” (2020) Front Med. <doi: 10.3389/fmed.2020. 00420>. Lactoferrin could treat or prevent such secondary bacterial infections. A variety of different possible mechanisms for lactoferrin's antibacterial effects have been proposed. See Barber et al, “Antimicrobial Functions of Lactoferrin Promote Genetic Conflicts in Ancient Primates and Modern Humans” PLoS Genetics (2016)<doi: 10.1371/journal.pgen.1006063>. For example, lactoferrin is an iron-binding protein and could act as an antibacterial defense mechanism by depleting the iron needed for bacterial growth. This invention encompasses any possible mechanism for lactoferrin's antibacterial efficacy. Lactoferrin could be effective against a wide variety of bacterial pathogens, including Streptococcus pneumoniae, Haemophilus influenzae, Pseudomonas aeruginosa, Staphylococcus aureus, group A streptococcus, and bacteria responsible for hospital-acquired bacterial infections.

Immune System Modulation by Lactoferrin: Lactoferrin is known to have effects on the immune system. For example, lactoferrin acts as an inhibitor of eosinophil migration and proliferation. In particular, lactoferrin's ability to inhibit eosinophil migration to the lung and respiratory tract could be useful in this invention. Lactoferrin also has similar effects on neutrophils. By this manner, lactoferrin may be effective in reducing the disease damage caused by viral infections by modulating the immune system.

Respiratory Viruses Cause Immune Overactivation: As the host innate immune system battles infections, there is elevated production of various cytokines and type I interferons (IFNs). See Shah et al, “Overview of Immune Response During SARS-CoV-2 Infection: Lessons From the Past” (2020) Front Immunol. <doi: 10.3389/fimmu.2020.01949>. In the case of prolonged infection, hyper-activation of the immune system may also result in the development of a pro-inflammatory microenvironment, leading to adverse outcomes. The induction of numerous pro-inflammatory lymphokines, such as IL-6, IL-1β, TNF-α, and CCL2, has been seen in COVID-19 cases. Thus, hyper-inflammation resulting from an unbalanced action of the immune system could exacerbate COVID-19 outcomes. The immune modulation effects of lactoferrin may also be effective in reducing the hyper-inflammation that sometimes occur with viral infections.

Excess Immune Response Caused by Vaccines: Lactoferrin may also be effective in reducing the immune-related adverse effects caused by antiviral vaccinations (e.g. eosinophil migration and proliferation). There is considerable concern about whether SARS-CoV-2 exposure postvaccination would cause eosinophil-associated lung pathology through immune hyper-activation. Administering lactoferrin after or prior to vaccination may be helpful in mitigating any excess immune response caused by the vaccine.

### Experimental Work

To validate the inventions described herein, experimental work was performed in the following manner. See publication by Medina-Magues et al, “Biological Cloth Face Coverings—The Reduction of SARS-CoV-2 and Influenza (H1N1) Infectivity by Viruferrin™ Treatment” (April 2021) Materials 14:2327<https://doi.org/10.3390/ma14092327>. This article is incorporated by reference herein.

### Effect of pH

Cell & Virus Preparations: Incubate Vero E6 cells in supplemented culture medium at 37° C. in 5% CO₂. Make SARS-CoV-2 (strain COV2019 Italy/INM11) virus stock at 100 TCID₅₀/mL (median tissue culture infectious dose). Lactoferrin Preparation: Make a stock solution of lactoferrin by dissolving lactoferrin in water at a concentration of 10 mg/mL. Adjust pH to the experimental protocol.

Cell Infection Mixture: Make serial two-fold dilutions of lactoferrin solution (starting from 10 mg/mL) and at various pH levels of pH 4, 5, 6, and 7 (adjusted by HCl 0.1 M). Add SARS-CoV-2 to each test sample. Infect the culture of Vero cells by adding the virus/lactoferrin mixture to the cell culture wells. Incubate for 1 hours at 37° C. in 5% CO₂. Cytopathic Effect by Virus: Examine the cells under microscope to detect cytopathic effect. The last dilution of the virus/lactoferrin fluid sample showing 50% of cytopathic effect over the cell layer is the EC₅₀ value. Cytotoxicity Test: To rule out cellular cytotoxicity by lactoferrin itself, a solution of 10 mg/mL lactoferrin was added to Vero cells and incubated up to 72 hours. The cells were then examined for any toxic effect. No cellular cytotoxicity was seen at this concentration of 10 mg/mL.

Pre-Exposure: In another experiment, pre-incubate the cells in various concentrations of lactoferrin (10 mg/ml, 5 mg/ml, 2.5 mg/ml, 1.25 mg/ml, 0.625 mg/ml and 0.313 mg/ml) for one hour. Then add the virus to the cells. Examine the cells under microscope to detect cytopathic effect.

Results: There was no inhibitory effect seen at any pH without the pre-incubation step. However, pre-incubation with lactoferrin at different concentrations showed an inhibitory effect on viral infection in Vero cells, mainly in a dose-dependent manner. There were exceptions to this at the extremes of doses, namely the higher dose (10 mg/mL) and the two lowest doses of 0.613 and 0.313 mg/mL.

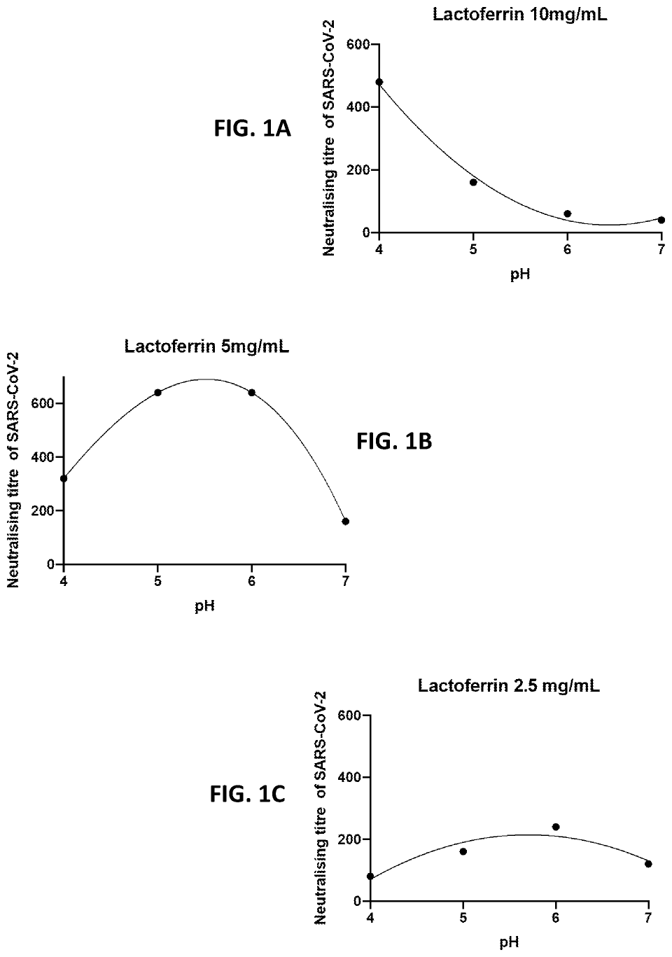

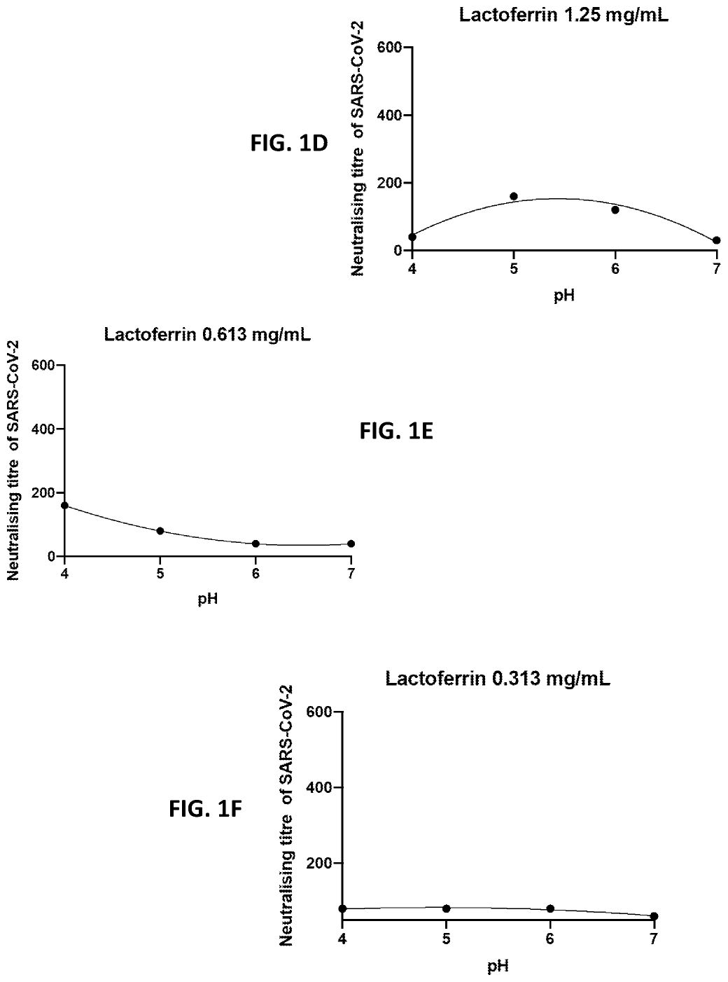

FIGS. 1A-1F show the viral inhibitory effect of various concentrations of lactoferrin across different pH levels. FIG. 1A shows the inhibitory effect at the highest lactoferrin concentration of 10 mg/mL. FIGS. 1B-1E shows the inhibitory effect at intermediate concentrations. FIG. 1B is 5 mg/mL; FIG. 1C is 2.5 mg/mL; FIG. 1D is 1.25 mg/mL; FIG. 1E is 0.613 mg/mL. FIG. 1F shows the lowest concentration at 0.313 mg/mL. As seen here, lactoferrin demonstrates an inhibitory effect in a dose-dependent manner, except for the extremes of doses, namely the higher dose (10 mg/mL) and the two lowest doses of 0.613 and 0.313 mg/mL. The maximum inhibitory effect was at 5 mg/mL in pH 4, 5, and 6. There was consistent low efficacy at pH 7. At the highest concentration of lactoferrin (10 mg/mL), a reduction in neutralizing titer was seen from pH 4 to 7, with an average titer of 452 to 40, respectively. This pH relationship occurred in a significant stepwise fashion.

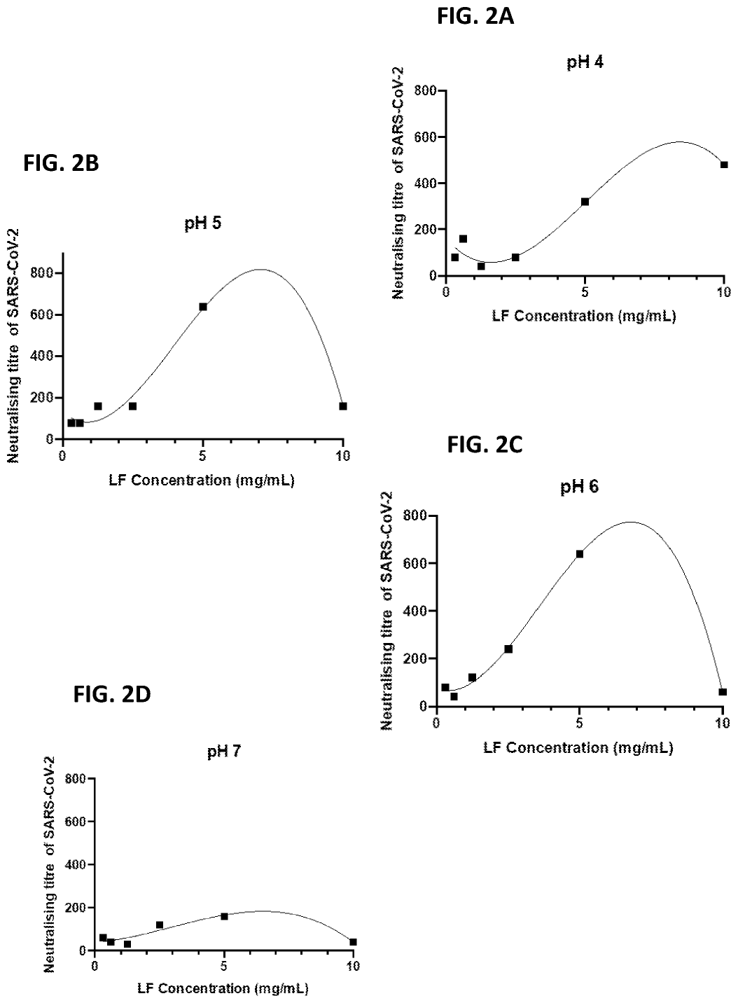

FIGS. 2A-2D show the viral inhibitory effect at various pH levels across different concentrations of lactoferrin exposure. FIG. 2A shows the results at pH 4; FIG. 2B shows the results at pH 5; FIG. 2C shows the results at pH 6; FIG. 2D shows the results at pH 7. At low concentrations of lactoferrin, there was consistently low antiviral efficacy. At a given pH, the trend was loss of antiviral efficacy with decreasing lactoferrin concentrations. However, at pH 4 and pH 7, antiviral efficacy increased at lactoferrin concentrations of 0.613 and 0.313 mg/mL; and for pH 6 only the lowest lactoferrin concentration of 0.313 mg/mL showed an increase. In general, the best efficacy was at the higher concentrations of 5 and 10 mg/mL. However, for pH 5, 6, and 7, the maximum concentration of lactoferrin (10 mg/mL) had less inhibitory effect than at the lower dose of 5 mg/mL. In general, a higher level of inhibitory activity was seen with pH 5 and pH 6 than with pH 4 and pH 7.

### Fabric Testing

Cell & Virus Preparations: Use H1N1 influenza virus (isolate California/04/2009) and SARS-CoV-2 (isolate USA-WA1/2020). Make virus stocks to titer at TCID₅₀. Use Vero E6 cells propagated in culture medium. Washout fluid from fabric samples (which contains eluted virus) could be tested for active virus quantity using either of two methods as follows. Plaque Count to Quantify Virus: Make culture plates of Vero E6 cells in 96-well plates and incubate at 37° C. and 5% CO₂ for 24 or 48 hours, respectively. Serially dilute the fabric washout fluid (containing eluted virus) and add to cell plates. Incubate infected cells for one hour at 37° C. and 5% CO₂. Then add a carboxymethyl-cellulose overlay. Incubate further for 36 hours at 37° C. and 5% CO₂. Discard the overlay. Fix the plates with an acetone, methanol, and acetic glacial acid mixture. Wash the plates and then add the primary antibody, which is either anti-SARS-CoV-2 antibody (monoclonal recombinant human IgG1) or anti-H1N1 influenza antibody (monoclonal anti-NP protein). Incubate overnight at 4° C. Wash off excess primary antibody and then add the secondary HRP-conjugated antibody. Incubate for 2 hours at 37° C. and then wash off the plates. Expose the plaques with a chromogen/peroxidase substrate. Count plaques using automated software.

MN Assay to Quantify Virus: Perform a microneutralization (MN) cytopathic effect (CPE) assay to quantify the amount of viable virus. Same as above, serially dilute the fabric washout fluid (containing eluted virus) and add to Vero E6 cell plates. Incubate the infected cells for 72 hours at 37° C. and 5% CO₂. Perform microscopic visualization to detect cytopathic effect of the virus-infected cells. The last dilution of the compound showing 50% of CPE over the cell layer is the EC₅₀ value.

Viruferrin-Treated Fabrics: Viruferrin™ is a proprietary formulation of bovine lactoferrin that can be used to treat fabrics. Viruferrin is made with bovine lactoferrin in spray-dried powder with >95% purity. Make a stock solution of Viruferrin by thoroughly mixing the bovine lactoferrin powder into water that has been adjusted to pH 5 using glacial acetic acid. Use amounts that make 1.0 gram/L solution of lactoferrin. This stock solution is used to treat the experimental fabrics, i.e. Viruferrin-coated fabrics.

FRESH FABRIC TESTING: This experiment tested fresh (not laundered) fabrics. Of the 160 total samples, 80 were control and 80 were Viruferrin-treated. Within each of those groups, one subgroup was tested with saliva and the other without. To simulate fouling of fabric from user breath and saliva, apply saliva onto some of the fabric samples. Perform this by dropwise application of 500 μL reconstituted test saliva onto one face of the fabric. As an additional positive control, testing was also done on growth media pus virus, but no fabric sample. Sample fabric materials (plain cotton control fabric and Viruferrin-coated fabric) were prepared for equal size (20 mm×20 mm) and weight (0.40 grams).

Drop Method for Virus Application: Allow fabric samples to dry and place in vials. Have preparations of SARS-CoV-2 or H1N1 virus at 1×10⁷ PFU/mL. Apply 200 μL of the virus preparations dropwise onto one side of the fabric (on the opposite side of saliva in the relevant groups). Seal the fabric vials and incubate at room temperature for a series of time durations (0, 1, 5, 15, 30, 60, 120, 360, 720, and 1440 min). After incubation, resuspend fabric samples in 500 μL of media and mix thoroughly. Elute the washout fluid and use the plaque count method above to quantify the amount of virus in the washout fluid.

Laundry Wash Testing: For the wash testing, launder the Viruferrin-treated fabrics at 30° C. with nonbiological washing detergent in a standard household washing machine on a 9-minute cycle. Repeat this washing 5 and 10 times. For controls, also have non-washed fabric samples. Wipe Method for Virus Application: Dilute 200 μL of the SARS-CoV-2 virus preparation in 2 mL of culture media. Evenly spread the virus into individual wells on a 9-well plate. Wipe each fabric sample across an individual well in a single swipe motion. Thus, the fabric samples collect virus from the wells. Determine the viral titer remaining in each well after the wiping action by MN assay describe above. This will inform the amount of virus that was transferred to the fabric sample.

Control for Cell Sensitivity to Viruferrin: Also perform control testing to rule out the possibility that the Viruferrin itself might be toxic to the cells. Prepare untreated control samples (plain cotton fabric) and Viruferrin-treated samples (Viruferrin-coated fabric) with fabrics of equal size (20 mm×20 mm) and weight (0.40 grams). Place fabric samples in vials and add 500 μL culture media (used as washout solution). Mix thoroughly and then elute the washout fluid into new sterile test tubes. Add virus to the test tube (either 50 μL of SARS-CoV-2 or 50 μL of H1N1 at a concentration of 1.0×10⁷ plaque forming units (PFU)/mL). Incubate for 30 minutes at 25° C. and then make serial dilutions to determine the infectious titer by TCID₅₀. Assess for effect by comparing the geometric mean titer (GMT) of the fabric samples.

Results: In the cell sensitivity control experiment, infectious titers were determined by TCID₅₀ for both H1N1 and SARS-CoV-2. GMTs for H1N1 and SARS-CoV-2 that were eluted from both the control and Viruferrin-treated fabric had identical GMTs. This indicates that Viruferrin treatment itself did not cause cell toxicity, i.e. that the antiviral effects of Viruferrin are not because of direct cell cytotoxicity.

Fresh Fabric Testing, H1N1: These are the results for H1N1 influenza A inactivation by Viruferrin-treated fabric. According to GMT, as compared to untreated cotton fabric samples, both the saliva-treated and non-saliva Viruferrin-treated fabrics reduced viral titers. The amount of active H1N1 in Viruferrin-treated fabrics was reduced at all timepoints, in both (+)saliva and (−)saliva treated fabrics. In Viruferrin-treated fabrics (−)saliva, there was reduction in viral titer at all timepoints up to 1440 minutes. There was no detectable viral titers in the washout from the Viruferrin-treated fabrics after the 360 minute timepoint. In Viruferrin-treated fabrics (+)saliva, there was reduction in viral titer at all timepoints up to 1440 minutes. There was no detectable viral titers in the washout from the Viruferrin-treated fabrics after the 120 minute timepoint.

Percent reduction in virus amount was also calculated comparing Viruferrin-treated versus untreated fabric. In Viruferrin-treated fabrics (−) saliva, there was an initial reduction of 92.59% and 98.92% at timepoint zero and 1 minute, respectively. After 5 minutes, there was 99.45% reduction in viral titer. In Viruferrin-treated fabrics (+) saliva, there was a 75.24% reduction after 1 minute, and 93.16% reduction after 5 minutes. After 60 minutes, there was a 99.8% reduction and then undetectable at 120 minutes.

Antiviral activity were also calculated from the GMT results. Viruferrin-treated samples (+) saliva showed initial antiviral activity of 0.2779 at timepoint zero and increased to 1.501 after 15 minutes of exposure. Antiviral activity increased with longer exposure times and reached 2.903 after 120 minutes. Viruferrin-treated samples (−) saliva showed an initial antiviral activity at timepoint zero of 0.4268 and reached 1.559 at 5 minutes. Antiviral activity increased with exposure time and reached 2.79 after 120 minutes.

Fresh Fabric Testing, SARS-CoV-2: These are the results for SARS-CoV-2 inactivation by Viruferrin-treated fabric. According to GMT, as compared to untreated cotton fabric samples, both (+)saliva and (−)saliva Viruferrin-treated fabric reduced viral titers at timepoints zero and 1 minute. In Viruferrin-treated fabrics (+)saliva, there was a reduction in viral titer for all timepoints (but not statistically significant). Percent reduction in virus amount was also calculated comparing Viruferrin-treated versus untreated fabric. In Viruferrin-treated fabrics (−) saliva, there was an initial reduction of 83.1% at timepoint zero. After 5 minutes, there was a 99.4% reduction in viral titer. In Viruferrin-treated fabrics (+)saliva, there was an initial reduction of 23.15% at timepoint zero. After 15 minutes, there was 96% reduction. In both (−) saliva and (+)saliva, virus was undetectable after 360 minutes.

Antiviral activity values were also calculated from the GMT results. Viruferrin-treated samples (+)saliva showed antiviral activity of 1.308 at 15 minutes of exposure. Antiviral activity values increased with longer exposure times and reached 2.838 after 720 minutes. Viruferrin-treated samples (−)saliva showed an initial antiviral activity value at timepoint zero of 0.4466 and reached 2.055 after 15 minutes. Antiviral activity increased with longer exposure times and reached 3.140 after 360 minutes.

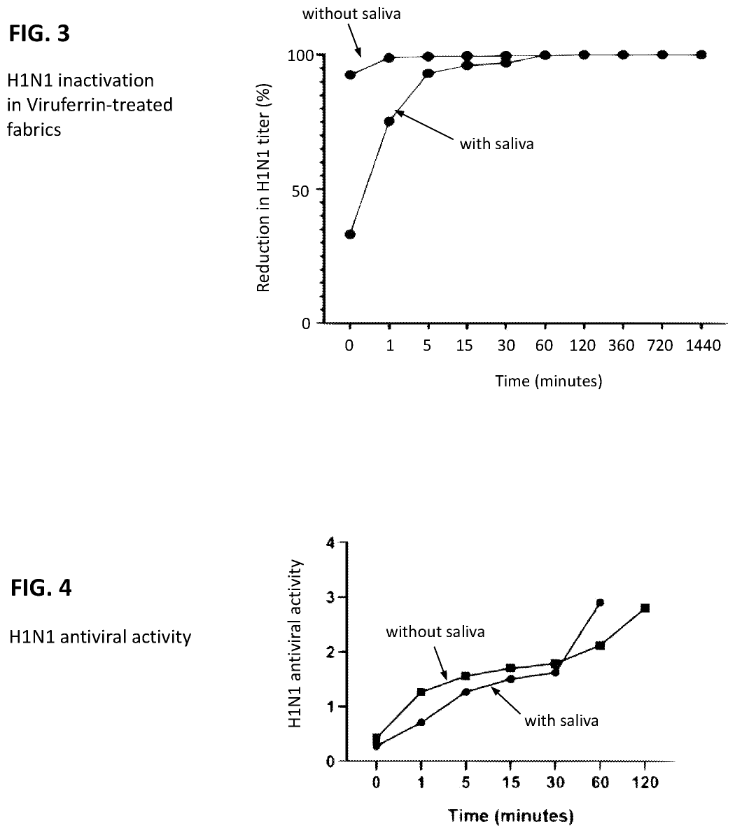

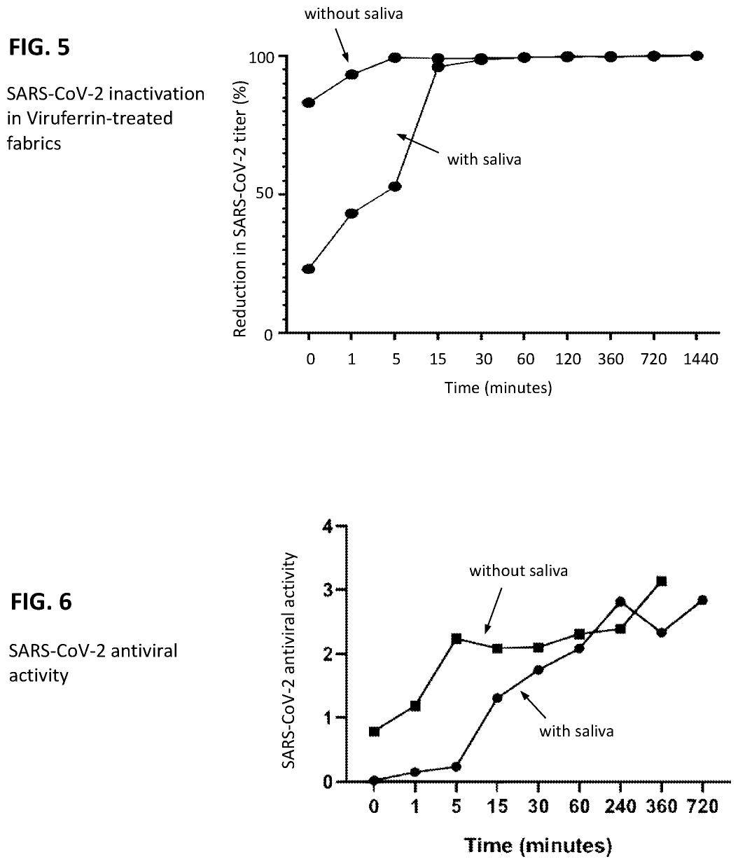

FIG. 3 shows the cumulative percentage reduction of H1N1 titers from Viruferrin-treated fabric samples collected at 10 time points, ranging from 0 to 1440 minutes (24 hours). Virus percent reduction was calculated in relation to virus recovered from the positive control. FIG. 4 shows the antiviral activity against H1N1 by Viruferrin-treated fabric samples collected at various time points. Antiviral activity values were calculated in relation to the cotton fabric control at timepoint zero. FIG. 5 shows the cumulative percentage reduction of SARS-CoV-2 titers from Viruferrin-treated fabric samples collected at 10 time points, ranging from 0 to 1440 minutes (24 hours). Virus percent reduction was calculated in relation to virus recovered from the positive control. FIG. 6 shows the antiviral activity against SARS-CoV-2 by Viruferrin-treated fabric samples collected at various time points. Antiviral activity values were calculated in relation to the cotton fabric control at timepoint zero.

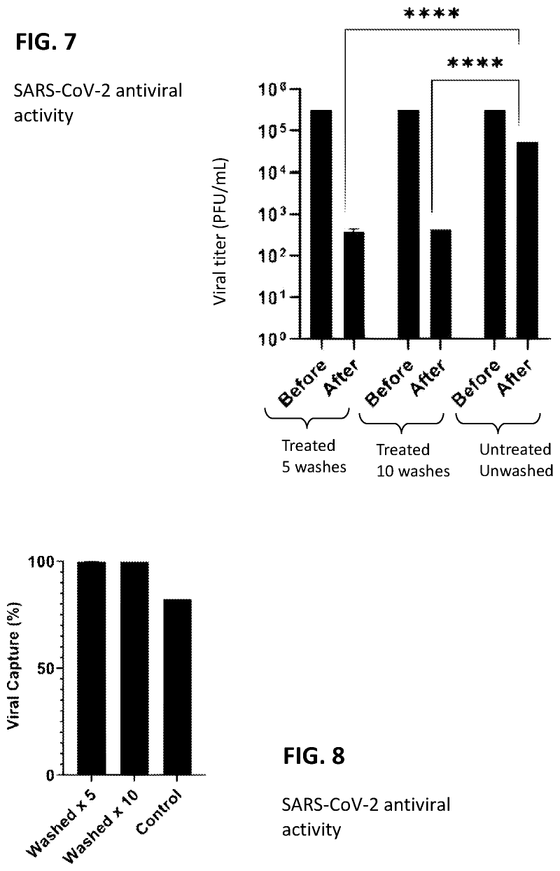

LAUNDRY WASH TESTING, SARS-COV-2: The percentage of viral capture was 99.9% for both the 5× and 10× washed Viruferrin-treated fabric materials. This is compared to 82% for the unwashed (fresh) and untreated (no Viruferrin) control fabrics. In FIGS. 7 and 8, the fabric samples were wiped in a single action in the selected well to pickup virus. FIG. 7 shows the viral titer of each well (virus remaining after the fabric wipe) using the microneutralization CPE-based assay. The bars represent GMTs. “Before” means prior to the fabric wipe, i.e. starting inoculum. “After” means after the fabric wipe. Data was analyzed for significance using a 2-way ANOVA with Sidak's multiple comparisons test (α=0.05, p ****≤0.0001) comparing Viruferrin-treated and the control fabric samples. FIG. 8 shows the capture rate of virus expressed as a percentage based upon the viral titer of the starting inoculum.

Discussion: This experimental work demonstrates that lactoferrin-treated fabric materials neutralize H1N1 influenza virus and SARS-CoV-2 virus that come in contact with the fabric. Against both viruses, contact with Viruferrin-treated fabric without saliva resulted in >99% reduction in viral titers as early as 5 minutes post-exposure. The presence of saliva had a significant effect on the antiviral capabilities for lactoferrin (a reduction in effectiveness from 92.6% to 33% with H1N1 and from 83% to 23% with SARS-CoV-2, both upon immediate contact). However, we believe that real world use will have substantially less saliva fouling compared to the large amount of saliva that was smeared onto the fabrics in the experiments.

But even with saliva fouling, antiviral potency increased with contact time and the saliva effect was fully overcome after sufficient exposure time. The plain cotton fabric materials also demonstrated some antiviral efficacy. But this is probably due to the loss of virus during the process of absorption and elution from fabric material, and also because virus viability naturally decreases over time. For both H1N1 and SARS-CoV-2, viral particles were undetected (detection limit of <1000 PFU) after 60 and 120 minutes with saliva, and 720 and 360 minutes without saliva. This experimental work also demonstrated the durability of the lactoferrin treatment on the fabric material. There was continued antiviral effect after 10 laundry washes.

### Cyclodextrin Testing

Culture Vero E6 cells in 96-well plates and then add standard amount of SARS-CoV-2 preparation. Add one of lactoferrin alone at 10 mg/mL, cyclodextrin alone at 10 mg/mL, and combination of lactoferrin (5 mg/mL) and cyclodextrin (10 mg/mL). Adjust to pH 7. Examine the cells under microscope to detect cytopathic effect. The last dilution of the virus fluid sample showing 50% of cytopathic effect over the cell layer is the EC₅₀ value. As a control without virus, neither the lactoferrin alone (10 mg/mL) or the cyclodextrin alone (10 mg/mL) had a cytopathic effect on the cells. In the virus-infected cells, none of the experiments showed antiviral activity. In particular, lactoferrin alone at 10 mg/mL did not show antiviral activity. And lactoferrin (5 mg/mL) in combination with cyclodextrin (10 mg/mL) did not show antiviral activity. This may indicate that lactoferrin must be used alone (without any other additives) to have antiviral activity. Other such additives may interfere with the antiviral effect of lactoferrin.

### Manufacturing

The following is a working example of how a snood could be made. The fabric is made of viscose 96% (rayon)/Lycra® 4% (polyether-polyurea copolymer). Dye the fabric with Novacron® black reactive dye. Print logo on dyed black fabric. Use bovine lactoferrin in spray-dried powder with >95% purity. Make a stock preparation of lactoferrin at 1.0 gram/L concentration by adding the bovine lactoferrin powder in water, which has been adjusted to pH 5.0-5.5 with glacial acetic acid. Mix thoroughly with an ultrasound mixer. Apply the lactoferrin mixture onto the fabric by padding, dipping, or spraying. Use an amount of 1.0 gram lactoferrin in 1.0 L aqueous solution covering 1.0 m² of the fabric, i.e. 1.0 gram lactoferrin per 1.0 m² of the fabric. Or expressed equivalently on a larger scale; 1,000 grams lactoferrin in 1,000 L aqueous solution covering 1,000 m² of the fabric. Dry the fabric at a temperature≤80° C. (e.g. a temperature in the range of 50-80° C.). Roll, cut, and package fabric snoods into individual units. Working examples of these snoods were made using this process.

The descriptions and examples given herein are intended merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not confined to any particular order of performance. Modifications of the disclosed embodiments incorporating the spirit and substance of the invention may occur to persons skilled in the art, and such modifications are within the scope of the invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

1. A breathable biological face covering comprising:
a fabric material;
an anti-pathogen substance on the fabric material, wherein the anti-pathogen substance comprises a lactoferrin protein.

2. The face covering of claim 1, wherein the amount of lactoferrin protein is 0.6-1.5 grams/m2 of fabric material.

3. The face covering of claim 1, wherein the lactoferrin protein is a bovine lactoferrin protein.

4. The face covering of claim 1, further comprising a fabric dye that comprises a reactive dye compound that bonds to the fabric material.

5. The face covering of claim 4, wherein the reactive dye compound also bonds to the lactoferrin protein.

6. The face covering of claim 4, wherein the reactive dye compound is anionic.

7. The face covering of claim 4, wherein the reactive dye compound comprises one or more sulfonic acid groups.

8. The face covering of claim 4, wherein the reactive dye compound is an azo dye compound having the diazo functional group (—N═N—).

9. The face covering of claim 1, wherein the face covering is a facemask or snood.

10. A method of making a breathable biological face covering, comprising:
having a fabric material;
applying lactoferrin to the fabric material, wherein the lactoferrin is provided in an aqueous solution containing lactoferrin at a concentration of 0.6-1.5 grams per liter.

11. The method of claim 10, wherein the resulting amount of lactoferrin applied to the fabric material is in the range of 0.6-1.5 grams/m2 of fabric material.

12. The method of claim 10, further comprising dyeing the fabric material with a fabric dye that comprises a reactive dye compound that bonds to the fabric material.

13. The method of claim 12, wherein the reactive dye compound also bonds to the lactoferrin protein.

14. The method of claim 12, wherein the reactive dye compound is anionic.

15. The method of claim 12, wherein the reactive dye compound comprises one or more sulfonic acid groups.

16. The method of claim 12, wherein the reactive dye compound is an azo dye compound having the diazo functional group (—N═N—).

17. A method of protecting against or treating for respiratory microbial pathogens, comprising:
having a lactoferrin composition comprising:
(a) aqueous fluid at pH<7.0;
(b) lactoferrin protein mixed in the aqueous fluid at a concentration of 0.6-1.5 grams per liter;
administering the lactoferrin composition into the respiratory tract.

18. The method of claim 17, wherein the lactoferrin composition is deposited in an upper airway and a lower airway.

19. The method of claim 17, wherein the lactoferrin composition contains no other ingredients having a molecular weight of greater than 500 grams/mol.

20. The method of claim 17, wherein the respiratory microbial pathogen is an influenza virus or coronavirus.
